(19) United States
(12) Patent Application Publication
(10) Pub. No.: US 2024/0394877 A1
(43) Pub. Date: Nov. 28, 2024

(54) MEDICAL SUPPORT SYSTEM AND MEDICAL SUPPORT METHOD

(71) Applicant: OLYMPUS MEDICAL SYSTEMS CORP., Tokyo (JP)

(72) Inventors: Yuta KOBAYASHI, Tokyo (JP); Haruhiko SAKAYORI, Tokyo (JP)

(73) Assignee: OLYMPUS MEDICAL SYSTEMS CORP., Tokyo (JP)

(21) Appl. No.: 18/750,364

(22) Filed: Jun. 21, 2024

Related U.S. Application Data
(63) Continuation of application No. PCT/JP2021/048045, filed on Dec. 23, 2021.

(51) Int. Cl. G06T 7/00; G16H 10/60; G16H 50/20

(52) U.S. Cl. CPC G06T 7/0012; G16H 10/60; G16H 50/20; G06T 2207/10068; G06T 2207/30096

(57) ABSTRACT

A lesion information acquisition unit acquires lesion information related to a lesion detected from an endoscopic image by an image analyzing device. A skill evaluation criterion setting unit sets a skill evaluation criterion related to a treatment to be performed on the detected lesion based on the lesion information.

### CROSS-REFERENCE TO RELATED APPLICATIONS

This application is based upon and claims the benefit of priority from the International Application No. PCT/JP2021/048045, filed on Dec. 23, 2021, the entire contents of which are incorporated.

### BACKGROUND

### 1. Technical Field

The present disclosure relates to a medical support system and a medical support method for supporting evaluation of treatment skills.

### 2. Description of the Related Art

The shortage of physicians in recent years reduces opportunities for expert physicians to take time to educate young physicians, and it is difficult to appropriately evaluate skills of the young physicians. In particular, in order to evaluate treatment skills in endoscopy, it is necessary for an expert physician to confirm a situation in which a young physician is actually performing a treatment, but there is a situation in which a busy expert physician cannot secure time for training of a young physician.

JP 2017-86685 A discloses a technique of detecting a medicine or a treatment tool used in endoscopy from a frame image included in an endoscopic moving image by image recognition, and recording spraying of the medicine or performance of treatment. The technique disclosed in JP 2017-86685 A aims to objectively evaluate the quality of the endoscopy by grasping time lengths and a number of times required for spraying the medicine or performing the treatment.

Capability of accurately evaluating treatment skills of physicians is important not only from the viewpoint of training of young physicians but also from the viewpoint of risk management in medical facilities. Therefore, development of a technique for supporting evaluation of treatment skills is desired.

### SUMMARY

The present disclosure has been made in view of such a situation, and an object of the present disclosure is to provide a technique for supporting evaluation of treatment skills of a physician.

A medical support system according to an aspect of the present disclosure includes: one or more processors having hardware, wherein the one or more processors acquire lesion information related to a lesion detected from an endoscopic image by a computer and set a skill evaluation criterion related to a treatment to be performed on the detected lesion based on the lesion information.

Another aspect of the present disclosure is a medical support method including: acquiring lesion information related to a lesion detected from an endoscopic image by a computer; and setting a skill evaluation criterion related to a treatment to be performed on the detected lesion based on the lesion information.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing a configuration of a medical support system according to an embodiment;

FIG. 2 is a diagram showing functional blocks of a server device;

FIG. 3 is a diagram showing an example of an endoscopic image;

FIG. 4 is a sequence diagram showing a procedure for setting a skill evaluation criterion;

FIGS. 5A-5C are diagrams showing example tables;

FIG. 6 is a diagram showing an example of a table;

FIG. 7 is a diagram showing an example of a table;

FIG. 8 is a diagram showing an example of past implementation information;

FIG. 9 is a diagram showing an example of information to be displayed;

FIG. 10 is a diagram showing an example of an endoscopic image in which a treatment tool is captured;

FIG. 11 is a diagram showing an example of information to be displayed;

FIG. 12 is a diagram showing an example of information to be displayed;

FIGS. 13A-13C are diagrams showing example tables;

FIG. 14 is a diagram showing an example of a table;

FIGS. 15A and 15B are diagrams showing example tables;

FIG. 16 is a diagram showing an example of a screen to be displayed;

FIG. 17 is a diagram showing another example of a screen to be displayed; and

FIG. 18 is a diagram showing another example of a screen to be displayed.

### DETAILED DESCRIPTION

The disclosure will now be described by reference to the preferred embodiments. This does not intend to limit the scope of the present disclosure, but to exemplify the disclosure.

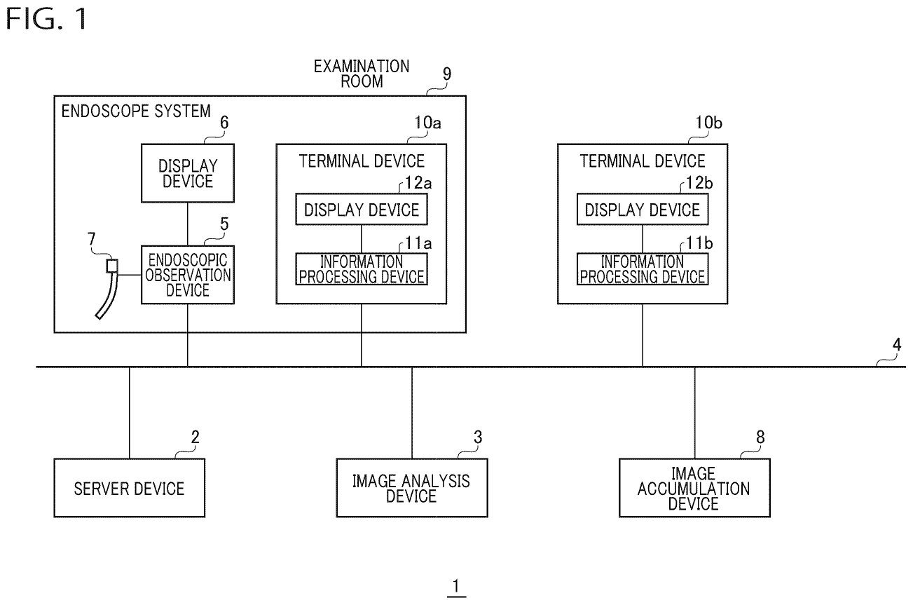

FIG. 1 shows a configuration of a medical support system 1 according to an embodiment. The medical support system 1 is provided in a medical facility such as a hospital that performs endoscopy. In the medical support system 1, a server device 2, an image analyzing device 3, an image accumulation device 8, an endoscope system 9, and a terminal device 10b are communicably connected via a network 4 such as a local area network (LAN). The endoscope system 9 is provided in an examination room, and includes an endoscopic observation device 5 and a terminal device 10a. In the medical support system 1, the server device 2, the image analyzing device 3, and the image accumulation device 8 may be provided outside the medical facility as a cloud server, for example.

The endoscopic observation device 5 is connected to an endoscope 7 inserted into a gastrointestinal tract of a patient. The endoscope 7 includes a light guide for transmitting illumination light provided from the endoscopic observation device 5 to illuminate an interior of the gastrointestinal tract. At a distal end of the endoscope 7, an illumination window for emitting the illumination light transmitted by the light guide to the living tissue and an imaging unit for imaging the living tissue at a predetermined cycle and outputting an imaging signal to the endoscopic observation device 5 are provided. The imaging unit includes a solid state image sensor (for example, a CCD image sensor or a CMOS image sensor) that converts incident light into an electric signal.

The endoscopic observation device 5 performs image processing on the imaging signal photoelectrically converted by the solid state image sensor of the endoscope 7 to generate an endoscopic image, and displays the endoscopic image on a display device 6 in real time. The endoscopic observation device 5 may have a function of performing special image processing for highlight display or the like in addition to normal image processing such as A/D conversion and noise removal. The endoscopic observation device 5 generates an endoscopic image at a predetermined cycle (for example, 1/60 seconds). The endoscopic observation device 5 may be configured by one or more processors having dedicated hardware, or may be configured by one or more processors having general-purpose hardware. The endoscope 7 of the embodiment is a flexible endoscope, and includes a forceps channel for inserting an endoscopic treatment tool. A physician can perform various endoscopic treatments during endoscopy by inserting and operating a treatment tool into the forceps channel.

The physician observes the endoscopic image displayed on the display device 6 according to the examination procedure. The physician observes the endoscopic image while moving the endoscope 7, and operates a release switch of the endoscope 7 when a lesion is shown on the display device 6. When the release switch is operated, the endoscopic observation device 5 captures (stores) the endoscopic image at the timing when the release switch is operated, and transmits the captured endoscopic image to the image accumulation device 8 together with information (image ID) for identifying the endoscopic image. Note that the endoscopic observation device 5 may collectively transmit a plurality of captured endoscopic images to the image accumulation device 8 after the examination. The image accumulation device 8 records the endoscopic image transmitted from the endoscopic observation device 5 in association with the examination ID for identifying the endoscopy. The endoscopic images accumulated in the image accumulation device 8 are used by a physician to create an examination report.

The terminal device 10a includes an information processing device 11a and a display device 12a, and is provided in an examination room. The terminal device 10a is used by a physician, a nurse, or the like to confirm information related to a treatment or information related to a lesion in real time during endoscopy. During endoscopy, the information processing device 11a acquires information related to a treatment and information related to a lesion from one or both of the server device 2 and the image analyzing device 3, and displays the acquired information on the display device 12a.

The terminal device 10b includes an information processing device 11b and a display device 12b, and is provided in a room other than the examination room. The terminal device 10b may be used, for example, when an expert physician confirms treatment skills of a young physician. The terminal devices 10a and 10b include one or more processors having general-purpose hardware.

The image analyzing device 3 is an electronic computational machine (computer) that analyzes an endoscopic image, detects a lesion included in the endoscopic image, and performs a differential diagnosis on the detected lesion. In the medical support system 1 of the embodiment, the endoscopic observation device 5 displays the endoscopic image from the display device 6 in real time and supplies the endoscopic image to the image analyzing device 3 in real time. The image analyzing device 3 has an artificial intelligence (AI) diagnosis function and may be configured by one or more processors having dedicated hardware, or may be configured by one or more processors having general-purpose hardware.

The image analyzing device 3 uses a learned model generated by machine learning using an endoscopic image for learning and information related to a lesion region included in the endoscopic image as teacher data. Annotation work of the endoscopic image is performed by an annotator having specialized knowledge such as a physician, and for machine learning, CNN, RNN, LSTM, or the like, which is a type of deep learning, may be used. When an endoscopic image is input, the learned model detects and outputs information related to a lesion (lesion information). The lesion information detected by the image analyzing device 3 may include a position of a contour of the lesion (contour coordinates), a size of the lesion, an invasion depth of the lesion, and a differential diagnosis result of the lesion. During the endoscopy, the image analyzing device 3 is provided with the endoscopic image from the endoscopic observation device 5 in real time, and supplies the detected lesion information to the server device 2 in real time.

The image analyzing device 3 of the embodiment also has a function of detecting a treatment content performed on a lesion by a physician (hereinafter, also referred to as “user”) by image analysis and outputting implementation information indicating the treatment content. For example, regarding the time when the user has performed the treatment (treatment time), the image analyzing device 3 determines a start of the treatment and starts counting of the treatment time when the treatment tool frames in the endoscopic image, and determines an end of the treatment and ends counting of the treatment time when the treatment tool frames out from the endoscopic image. Note that the image analyzing device 3 may determine the end of the treatment at the timing when the physician operates the release switch after the treatment tool is framed out from the endoscopic image and the treated part is washed with water. In this case, information on the operation of the release switch is provided from the endoscopic observation device 5 to the image analyzing device 3, and used for the determination of the end of the treatment. In this manner, the image analyzing device 3 may count the treatment time and output the implementation information including the treatment time to the server device 2.

When the treatment tool frames in the endoscopic image, the image analyzing device 3 specifies the type of the treatment tool by image analysis. Examples of the treatment tool include a high frequency treatment tool used to resect a lesion, such as a high frequency snare, an incision forceps, a high frequency knife, and the like. For example, a high frequency snare is used for endoscopic mucosal resection (EMR), and a high frequency knife is used for endoscopic submucosal dissection (ESD). Examples of the treatment tool may include forceps such as biopsy forceps, grasping forceps, and hot biopsy forceps, and an injection needle such as a biopsy needle and a local injection needle. When the type of the captured treatment tool is specified, the image analyzing device 3 outputs information indicating the specified type of the treatment tool to the server device 2 as implementation information.

The image analyzing device 3 detects an outline (resection edge) of a portion actually resected by the user with the treatment tool. When the user resects a lesion, the image analyzing device 3 detects a position removed by the treatment tool in real time and outputs implementation information including resection edge information indicating the position of the resection edge to the server device 2 in real time. Note that the image analyzing device 3 may output the resection edge information indicating the position of the resection edge to the server device 2 after the removal by the treatment tool is completed.

In addition, the image analyzing device 3 has a function of detecting a matter that has occurred during implementation of the treatment (resection of the lesion) as a treatment content. Specifically, the image analyzing device 3 may detect a degree of bleeding during implementation of the treatment and output implementation information including information indicating the degree of bleeding to the server device 2 after the end of the treatment. In addition, the image analyzing device 3 may monitor the occurrence of perforation during implementation of the treatment and output implementation information including information indicating whether or not perforation has occurred to the server device 2 after the treatment is completed. As described above, the image analyzing device 3 of the embodiment has a function of not only outputting lesion information detected by the AI diagnosis function to the server device 2, but also outputting implementation information indicating a treatment content performed on a lesion by the user to the server device 2. In the image analyzing device 3, the lesion information output function and the implementation information output function may be realized by separate processors.

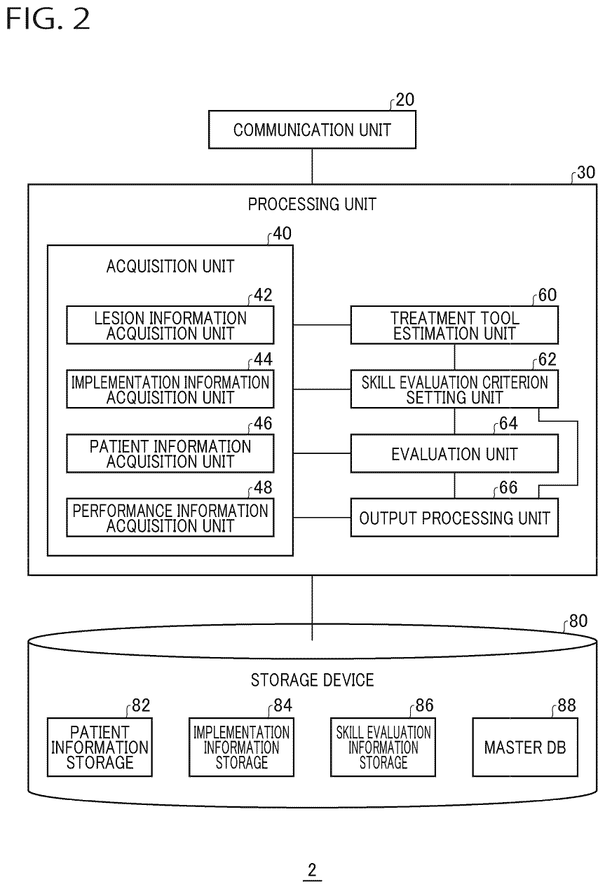

FIG. 2 shows functional blocks of the server device 2. The server device 2 has a function of supporting evaluation of treatment skills in endoscopy, and includes a communication unit 20, a processing unit 30, and a storage device 80. The communication unit 20 transmits and receives information such as data and instructions to and from the image analyzing device 3, the endoscopic observation device 5, the image accumulation device 8, the terminal device 10a, and the terminal device 10b via the network 4. The processing unit 30 includes an acquisition unit 40, a treatment tool estimation unit 60, a skill evaluation criterion setting unit 62, an evaluation unit 64, and an output processing unit 66, and the acquisition unit 40 includes a lesion information acquisition unit 42, an implementation information acquisition unit 44, a patient information acquisition unit 46, and a record information acquisition unit 48. The storage device 80 includes a patient information storage 82, an implementation information storage 84, a skill evaluation information storage 86, and a master DB 88.

The server device 2 includes a computer, and various functions shown in FIG. 2 are implemented by the computer executing a program. The computer includes, as hardware, a memory for loading a program, one or more processors for executing the loaded program, an auxiliary storage device, other LSIs, and the like. The processor includes a plurality of electronic circuits including a semiconductor integrated circuit and an LSI, and the plurality of electronic circuits may be mounted on one chip or may be mounted on a plurality of chips. It should be understood by those skilled in the art that the functional blocks shown in FIG. 2 are implemented by cooperation of hardware and software, and therefore, these functional blocks can be implemented in various forms by only hardware, only software, or a combination thereof.

The endoscopic observation device 5 of the embodiment supplies an endoscopic image to the image analyzing device 3, and the image analyzing device 3 performs image analysis on the supplied endoscopic image in real time to detect a lesion. The image analyzing device 3 detects a lesion, generates lesion information related to the lesion, and outputs the lesion information to the server device 2. The lesion information detected by the image analyzing device 3 may include the following information.
a location of the lesion (contour information)
a size of the lesion
invasion depth of the lesion
differential diagnosis result of the lesion

In the embodiment, the information indicating the position of the lesion may be position coordinates of a contour surrounding a bottom surface of the lesion having a three-dimensional shape, and the information indicating the size of the lesion may be the largest diameter of the bottom surface of the lesion. The information indicating the invasion depth of the lesion may be expressed by a hierarchical code of Tis, T1, T2, T3, T4a, and T4b using the TNM classification. The differential diagnosis result of the lesion may be information indicating whether the lesion is non-neoplastic or neoplastic.

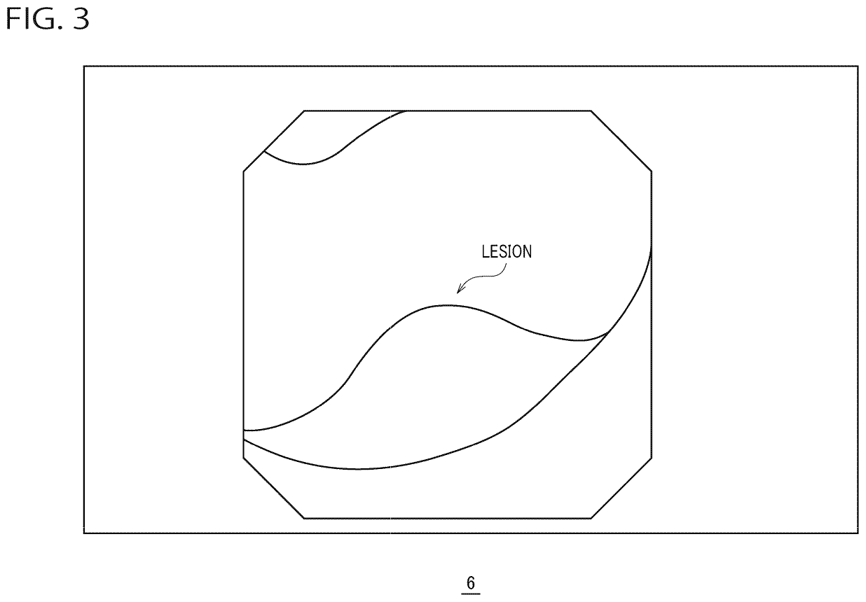

FIG. 3 shows an example of the endoscopic image displayed on the display device 6. In this endoscopic image, a raised portion is a lesion, and when detecting the lesion included in the endoscopic image, the image analyzing device 3 outputs lesion information to the server device 2. In the medical support system 1 of the embodiment, the server device 2 sets a criterion for evaluating the treatment skill of the lesion by the user (hereinafter referred to as “skill evaluation criterion”) based on the information output from the image analyzing device 3, and evaluates the treatment skill of the user based on the set skill evaluation criterion and the implementation information indicating the content of the treatment actually performed by the user. Hereinafter, a procedure for setting the skill evaluation criterion will be described.

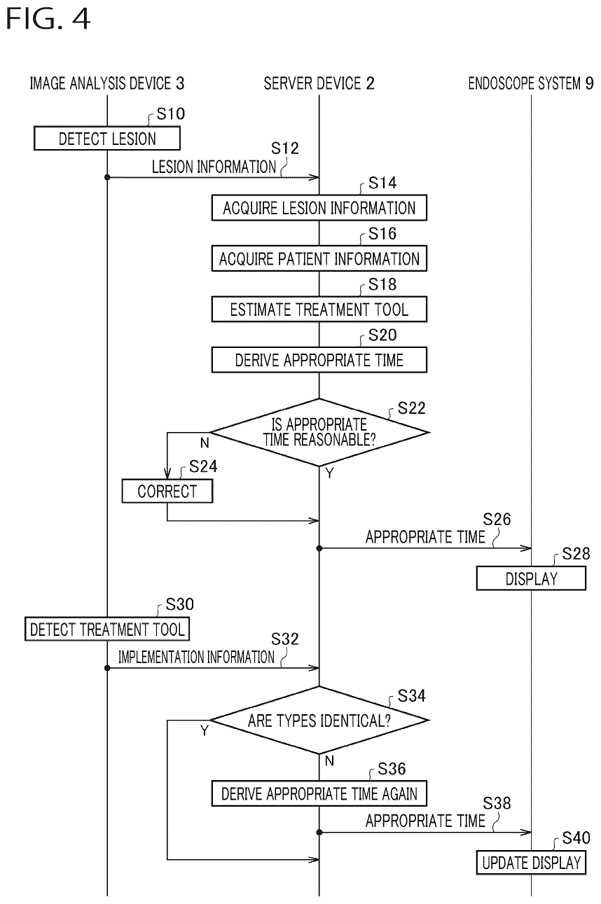

FIG. 4 is a sequence diagram showing a procedure for setting the skill evaluation criterion. When the image analyzing device 3 detects a lesion from the endoscopic image (S10), information on the lesion (lesion information) is output to the server device 2 (S12). In the server device 2, the lesion information acquisition unit 42 acquires the lesion information output from the image analyzing device 3 (S14). During the endoscopy, since the image analyzing device 3 performs lesion detection processing for each endoscopic image, when the same lesion is included in a plurality of consecutive endoscopic images, the same lesion is detected from all the endoscopic images. Therefore, in a case where a currently detected lesion is the same as a last detected last lesion, it is preferable that the image analyzing device 3 does not output lesion information on the same lesion to the server device 2. That is, the image analyzing device 3 may be controlled so as to output, when one lesion is first detected from the endoscopic image, lesion information on the lesion to the server device 2, and not to output the lesion information to the server device 2 if the same lesion is detected from the subsequent endoscopic image.

The skill evaluation criterion setting unit 62 sets a skill evaluation criterion related to a treatment to be performed on a detected lesion based on the lesion information acquired by the lesion information acquisition unit 42. In the embodiment, the skill evaluation criterion may include an appropriate time required for performing a treatment of a lesion and a resection line for resecting the lesion, and the skill evaluation criterion setting unit 62 may set the appropriate time and the resection line as the skill evaluation criterion based on the lesion information. In a sequence diagram shown in FIG. 4, the skill evaluation criterion setting unit 62 derives an appropriate time required for performing a treatment of a lesion based on the lesion information (S20). Note that steps S16 and S18 will be described later.

As described above, the lesion information provided from the image analyzing device 3 includes the position (contour coordinates) of the lesion, the size of the lesion, the invasion depth of the lesion, and the differential diagnosis result of the lesion. The skill evaluation criterion setting unit 62 may set the skill evaluation criterion based on at least one of the position (contour coordinates) of the lesion, the size of the lesion, the invasion depth of the lesion, and the differential diagnosis result of the lesion. Note that the skill evaluation criterion setting unit 62 may set the skill evaluation criterion based on at least two or more of the position (contour coordinates) of the lesion, the size of the lesion, the depth invasion depth of the lesion, or the differential diagnosis result of the lesion.

The skill evaluation criterion setting unit 62 derives an appropriate time required for performing a treatment of a lesion based on the lesion information (S20). In the embodiment, the skill evaluation criterion setting unit 62 derives an appropriate time by adding time corresponding to a weight score according to the lesion information to a base time (BT) required for performing a lesion treatment. The master DB 88 records weight scores corresponding to the lesion information.

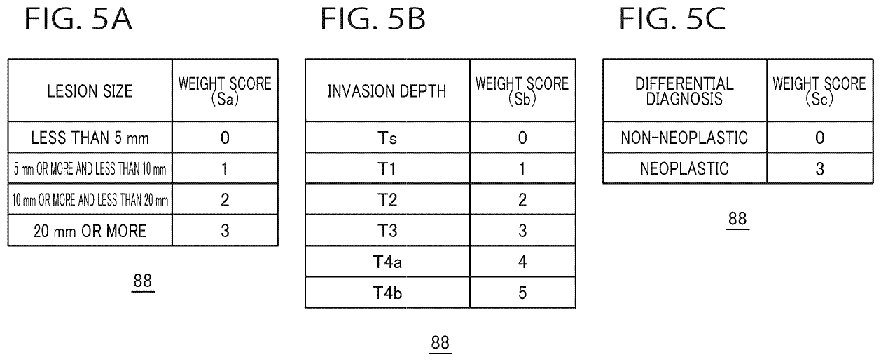

FIG. 5A shows an example of a table in which the size of the lesion and a weight score Sa are associated with each other. In this table, a weight score 0 is assigned to a lesion size of less than 5 mm, a weight score 1 is assigned to a lesion size of 5 mm or more and less than 10 mm, a weight score 2 is assigned to a lesion size of 10 mm or more and less than 20 mm, and a weight score 3 is assigned to a lesion size of 20 mm or more.

FIG. 5B shows an example of a table in which the invasion depth of the lesion and a weight score Sb are associated with each other. In this table, a weight score 0 is assigned to an invasion depth Ts, a weight score 1 is assigned to an invasion depth T1, a weight score 2 is assigned to an invasion depth T2, a weight score 3 is assigned to an invasion depth T3, a weight score 4 is assigned to an invasion depth T4a, and a weight score 5 is assigned to an invasion depth T4b.

FIG. 5C shows an example of a table in which the differential diagnosis result and a weight score Sc are associated with each other. In this table, a weight score of 0 is assigned to a non-neoplastic differential diagnosis and a weight score of 3 is assigned to a neoplastic differential diagnosis.

The skill evaluation criterion setting unit 62 may calculate the appropriate time required for performing lesion treatment using the following calculation formula (1).

$$Appropriate time = \text{} base time ( BT ) + Sa \times Ta + Sb \times Tb + Sc \times Tc \qquad ( 1 )$$

Here, Sa is a weight score corresponding to the lesion size, Sb is a weight score corresponding to the invasion depth, Sc is a weight score corresponding to the differential diagnosis result, Ta is an additional time related to the lesion size, Tb is an additional time related to the invasion depth, and Tc is an additional time related to the differential diagnosis result.

In the embodiment, the base time (BT) is 8 minutes, Ta is 30 seconds, Tb is 30 seconds, and Tc is 30 seconds. Note that Ta, Tb, and Tc may be different times. In the embodiment, it is assumed that the lesion information acquisition unit 42 acquires lesion information including a lesion size, an invasion depth, and a differential diagnosis result as in the following.
Lesion size: 15 mm
Invasion depth: T2
Differential diagnosis result: non-neoplastic

The skill evaluation criterion setting unit 62 derives the following weight scores with reference to the master DB 88.
Weight score Sa: 2
Weight score Sb: 2

Weight score Sc: 0
In this case, the skill evaluation criterion setting unit 62 calculates the appropriate time for a treatment from the calculation formula (1) as follows.

$$Appropriate time = 8 minutes + 2 \times 30 seconds + 2 \times 30 seconds + 0 \times 30 seconds = 10 minutes$$

In this manner, the skill evaluation criterion setting unit 62 may calculate the appropriate time required for performing the treatment of the lesion based on the lesion information.

Note that the skill evaluation criterion setting unit 62 may calculate the appropriate time in consideration of not only the lesion information but also the patient information of the endoscopy. In this case, the master DB 88 records weight scores corresponding to the patient information.

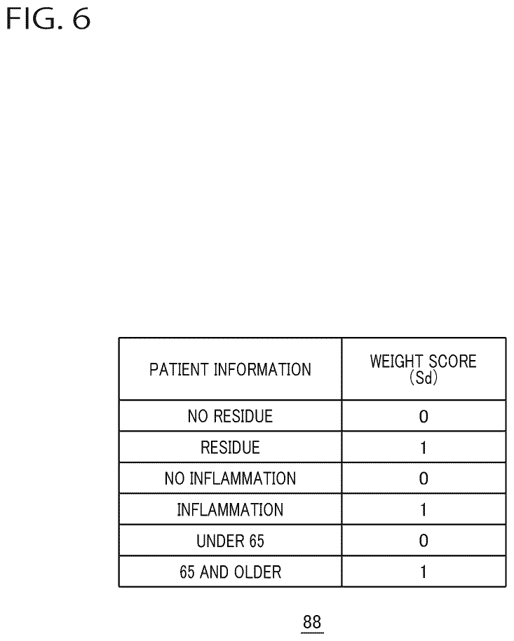

FIG. 6 shows an example of a table in which patient information and a weight score Sd are associated with each other. In this table, a weight score 0 is assigned to patient information “no residue”, a weight score 1 is assigned to patient information “residue”, a weight score 0 is assigned to patient information “no inflammation”, a weight score 1 is assigned to patient information “inflammation”, a weight score 0 is assigned to patient information “under 65 years old”, and a weight score 1 is assigned to patient information “65 years old and older”.

Before starting the endoscopy, the patient information acquisition unit 46 acquires patient information with reference to examination order information transmitted from a hospital information system (HIS) (S16). The skill evaluation criterion setting unit 62 may calculate the appropriate time based on the lesion information and the patient information. Note that, in the example shown in FIG. 6, the weight score Sd is assigned to each of the information related to the residue, the information related to the inflammation, and the information related to the age, but the weight score Sd may not be assigned to all of these three pieces of information, and may be assigned to at least one piece of information.

In the embodiment, the patient information acquisition unit 46 acquires patient information including at least one of the information related to residues, the information related to inflammation, and the information related to age, and the skill evaluation criterion setting unit 62 derives an appropriate time based on the lesion information and the patient information acquired by the patient information acquisition unit 46 (S20). At this time, the skill evaluation criterion setting unit 62 may calculate the appropriate time required for performing the lesion treatment using the following calculation formula (2).

$$Appropriate time = \text{} base time ( BT ) + Sa \times Ta + Sb \times Tb + Sc \times Tc + Sd \times Td \qquad ( 2 )$$

Here, Sa is a weight score corresponding to the lesion size, Sb is a weight score corresponding to the invasion depth, Sc is a weight score corresponding to the differential diagnosis result, Sd is a weight score corresponding to the patient information, Ta is an additional time related to the lesion size, Tb is an additional time related to the invasion depth, Tc is an additional time related to the differential diagnosis result, and Td is an additional time related to the patient information.

Further, the skill evaluation criterion setting unit 62 may calculate the appropriate time in consideration of the type of the treatment tool to be used. In this case, the master DB 88 records the weight score corresponding to the type of the treatment tool.

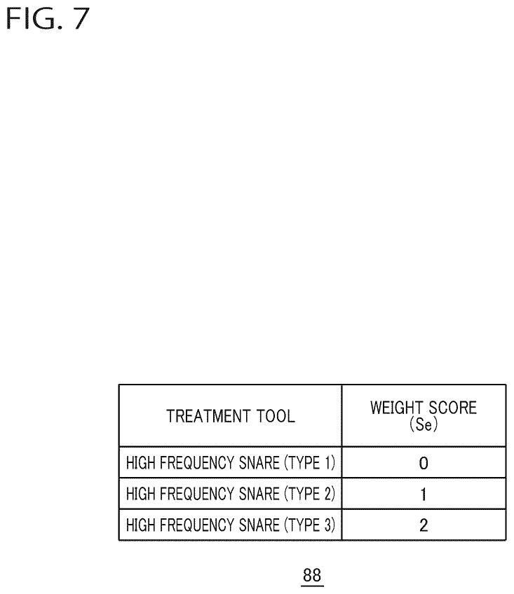

FIG. 7 shows an example of a table in which the type of the treatment tool and a weight score Se are associated with each other. In this table, a weight score 0 is assigned to the high frequency snare (type 1), a weight score 1 is assigned to the high frequency snare (type 2), and a weight score 2 is assigned to the high frequency snare (type 3). Note that the master DB 88 may have a table in which the types of all treatment tools that may be used in the treatment are associated with the weight score Se. The treatment tool estimation unit 60 estimates

and acquires the type of the treatment tool used to treat a lesion based on the lesion information (S18). For example, the treatment tool estimation unit 60 may estimate the type of the treatment tool to be used based on data obtained by statistically processing information on treatments performed in the past. The skill evaluation criterion setting unit 62 derives an appropriate time based on the lesion information and the treatment tool information related to the estimated type of the treatment tool (S20). At this time, the skill evaluation criterion setting unit 62 may calculate the appropriate time required for performing the lesion treatment using the following calculation formula (3).

$$Appropriate time = \text{} base time ( BT ) + Sa \times Ta + Sb \times Tb + Sc \times Tc + Sd \times Td + Se \times Te \qquad ( 3 )$$

Here, Sa is a weight score corresponding to the lesion size, Sb is a weight score corresponding to the invasion depth, Sc is a weight score corresponding to the differential diagnosis result, Sd is a weight score corresponding to the patient information, Se is a weight score corresponding to the type of the treatment tool, Ta is an additional time related to the lesion size, Tb is an additional time related to the invasion depth, Tc is an additional time related to the differential diagnosis result, Td is an additional time related to the patient information, and Te is an additional time related to the type of the treatment tool. Note that the treatment tool estimation unit 60 may exclude the parameter (Sd×Td) related to the patient information from the calculation formula (3).

As described above, the skill evaluation criterion setting unit 62 calculates an appropriate time for performing a treatment. In FIG. 4, before execution of the step of S20, the step of S14 is necessarily executed, but the steps of S16 and S18 may be executed as necessary.

When the treatment tool estimation unit 60 estimates that the treatment tool to be used is the high frequency snare (type 1), the weight score Se is “0”. Hereinafter, a case where the skill evaluation criterion setting unit 62 calculates the appropriate time as “10 min” using the calculation formula (3) will be described.

The implementation information storage 84 stores implementation information indicating past treatment contents implemented in the hospital facility. The implementation information stored in the implementation information storage 84 includes at least the type of the treatment tool that has been used and the implementation time of the treatment. The skill evaluation criterion setting unit 62 may determine whether or not the calculated appropriate time is reasonable by comparison with the calculated appropriate time with the past record stored in the implementation information storage 84 (S22). The record information acquisition unit 48 acquires, from the implementation information storage 84, information indicating past treatment contents using the treatment tool of the same type as the treatment tool estimated by the treatment tool estimation unit 60, and provides the information to the skill evaluation criterion setting unit 62.

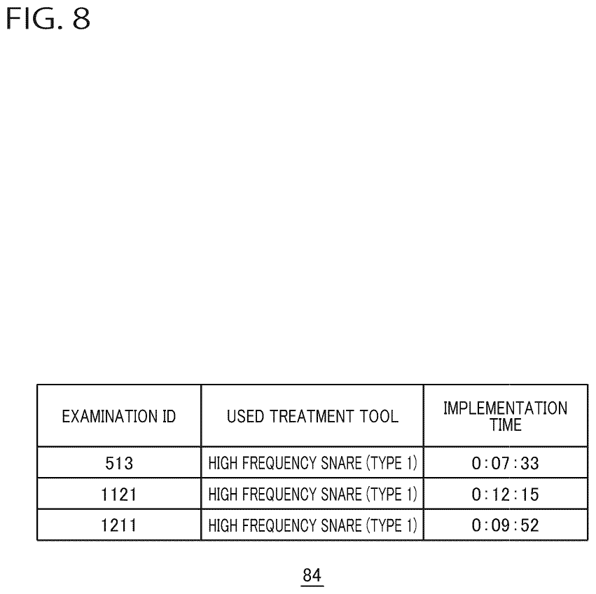

FIG. 8 shows an example of past implementation information read from the implementation information storage 84. FIG. 8 shows a list of the past implementation information using the high frequency snare (type 1). The skill evaluation criterion setting unit 62 determines whether or not the calculated appropriate time (10 minutes) is reasonable based on the implementation time in the past treatment using the same type of the treatment tool (S22). Here, the average of the three implementation times shown in FIG. 8 is 9 minutes 53 seconds, which is approximately equal to 10 minutes. Therefore, the skill evaluation criterion setting unit 62 determines that the calculated appropriate time is reasonable as compared with the past record (Y in S22). Note that, in a case where the calculated appropriate time is different from the past actual value (average value) by a predetermined time (for example, 5 minutes) or more, the skill evaluation criterion setting unit 62 preferably determines that the calculated appropriate time is not reasonable as compared with the past record (N in S22), and corrects the appropriate time to become reasonable (S24). In this manner, the skill evaluation criterion setting unit 62 may derive the appropriate time again based on the information indicating the past treatment contents. When the skill evaluation criterion setting unit 62 sets the appropriate time, the output processing unit 66 provides information indicating the appropriate time to the endoscope system 9 (S26). At this time, the output processing unit 66 may also provide the lesion information to the endoscope system 9.

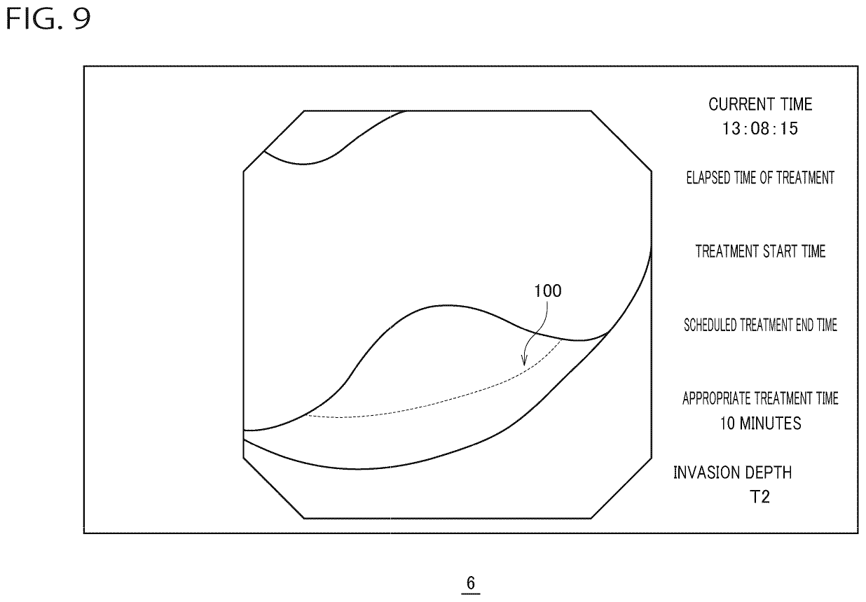

FIG. 9 shows an example of information displayed on the display device 6. The endoscopic observation device 5 displays information indicating appropriate treatment time on the display device 6 (S28). Note that, in the endoscope system 9, the information indicating the appropriate treatment time may be displayed on the display device 12a by the information processing device 11a. In any case, it is preferable that the information indicating the appropriate treatment time is displayed on a display device that can be viewed by the user who performs the treatment from now, and is presented as the reference information when the treatment is performed. As described above, the endoscopic observation device 5 or the information processing device 11a in the endoscope system 9 displays the appropriate treatment time derived based on the type of the treatment tool estimated to be used on the display device before the user starts a treatment of the lesion.

The endoscopic observation device 5 displays the information indicating the invasion depth on the display device 6 based on the lesion information provided from the server device 2. In a case where position coordinates of a line from which the lesion is to be removed are provided from the server device 2, the endoscopic observation device 5 may display a resection line 100 indicating the position of the line on the display device 6. As will be described later, in the server device 2, the skill evaluation criterion setting unit 62 sets the resection line 100 based on a lesion position (contour coordinates) included in the lesion information as one of the skill evaluation criteria. The resection line 100 is an ideal line when the user removes the lesion using the treatment tool, and the endoscopic observation device 5 displays the resection line 100 superimposed on the endoscopic image, so that the user can recognize that the lesion may be resected along the resection line 100. Note that, as shown in FIG. 9, it is preferable that the resection line 100 is displayed by being superimposed on the endoscopic image to support the resection of the lesion by the user, but the resection line 100 in the medical support system 1 only needs to be used as one of skill evaluation criteria, and it is not necessary to display the resection line superimposed on the endoscopic image.

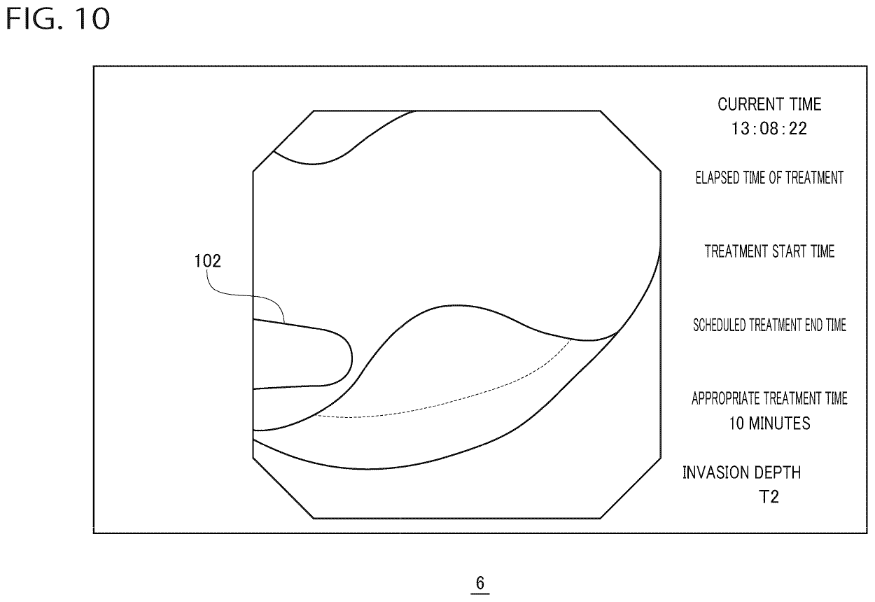

FIG. 10 shows an example of an endoscopic image in which the treatment tool is captured. When a treatment tool 102 is framed in the endoscopic image, the image analyzing device 3 detects the treatment tool 102 included in the endoscopic image and specifies the type of the treatment tool by image analysis (S30). Upon detection of the treatment tool 102, the image analyzing device 3 determines the start of the treatment and starts counting of the treatment time. The image analyzing device 3 provides the server device 2 with treatment tool information related to the identified type of the treatment tool and implementation information including a treatment implementation start time (S32). Here, the treatment implementation start time is 13:08:22.

In the server device 2, the implementation information acquisition unit 44 acquires the treatment tool information and the implementation information including the treatment implementation start time. The skill evaluation criterion setting unit 62 determines whether or not the type of the treatment tool estimated by the treatment tool estimation unit 60 matches the type of the treatment tool 102 specified by the image analyzing device 3 (S34). If the type of the treatment tool estimated here matches the type of the specified treatment tool 102 (Y in S34), the skill evaluation criterion setting unit 62 recognizes that it is not necessary to change the appropriate treatment time derived before the start of treatment. On the other hand, if the estimated type of the treatment tool does not match the type of the treatment tool 102 to be actually used (N in S34), the skill evaluation criterion setting unit 62 derives the appropriate time again based on the type of the treatment tool 102 to be actually used (S36). For example, when the estimated treatment tool is the high frequency snare (type 1) while the treatment tool actually specified is the high frequency snare (type 2), the skill evaluation criterion setting unit 62 derives the appropriate time again using the calculation formula (3), and the output processing unit 66 provides information indicating the corrected appropriate time to the endoscope system 9 (S38). Here, it is assumed that the skill evaluation criterion setting unit 62 has corrected the appropriate time to “12 minutes” using the calculation formula (3). Note that the output processing unit 66 provides the treatment start time (13:08:22) to the endoscope system 9 regardless of the presence or absence of correction of the appropriate time, and causes to start counting of the treatment time in the endoscope system 9.

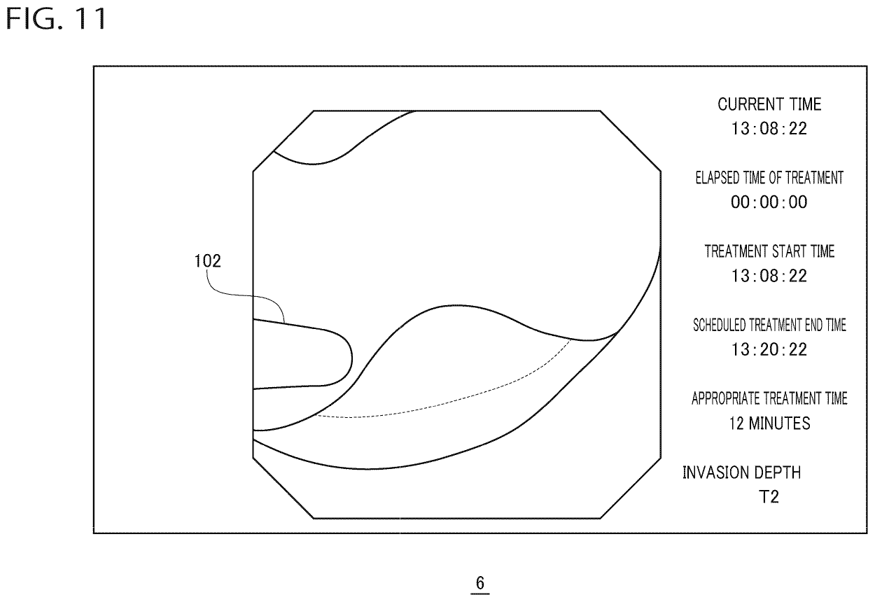

FIG. 11 shows an example of information displayed on the display device 6. The endoscopic observation device 5 displays information indicating the appropriate treatment time set based on the type of the treatment tool actually used on the display device 6 (S40). Note that, as described above, the information indicating the appropriate treatment time may be displayed on the display device 12a by the information processing device 11a. The display device 6 may display the scheduled treatment end time obtained by adding the appropriate treatment time to the treatment start time together with the information indicating the treatment start time. After the treatment start time (13:08:22), the endoscopic observation device 5 displays the elapsed time from the start of the treatment of the lesion as “elapsed time of treatment”. The user performs treatment with the aim that the elapsed time of treatment falls within the appropriate treatment time.

The above is a description of the procedure of setting the appropriate treatment time, which is one of the skill evaluation criteria, and as described above, the skill evaluation criterion setting unit 62 sets the resection line 100 for resecting a lesion as another skill evaluation criterion. The skill evaluation criterion setting unit 62 sets the resection line 100 that allows reliable resection of a lesion without unnecessary removal of the lesion based on the contour information of the lesion included in the lesion information. The resection line 100 is set to remove only a portion necessary for the lesion resection, and if a line actually cut by the user (resection edge) is along the resection line 100, the treatment skill will be highly evaluated, whereas if the resection edge is inside or outside the resection line 100, the treatment skill will be evaluated low.

When the user starts a treatment, the image analyzing device 3 detects a treatment content performed on a lesion by the user by image analysis, and outputs implementation information indicating the treatment content to the server device 2. The image analyzing device 3 detects a position actually resected by the user with the treatment tool 102 (resection edge). The image analyzing device 3 may detect a removal position in real time and output the resection edge information indicating the detected removal position to the server device 2 in real time as the implementation information, or may output the resection edge information indicating all the removal positions to the server device 2 after completion of the removal.

In the server device 2, the implementation information acquisition unit 44 acquires the implementation information output from the image analyzing device 3. The evaluation unit 64 determines whether or not the removal of the lesion is appropriately performed based on the skill evaluation criterion set by the skill evaluation criterion setting unit 62 and the implementation information acquired by the implementation information acquisition unit 44. Here, the evaluation unit 64 compares the position coordinates of the resection line 100 with the position coordinates of the resection edge actually removed by the user, and detects deviation between the resection line 100 and the resection edge by the user in real time. When the user removes a portion within the resection line 100, that is, when the resection edge by the user is located inside the resection line 100 and a portion to be removed remains, the evaluation unit 64 generates a warning and causes the output processing unit 66 to transmit the warning to the endoscope system 9. In the endoscope system 9, it is preferable that the endoscopic observation device 5 or the information processing device 11a output the warning indicating that the user removes a portion within the resection line 100 by voice or image to urge the user to perform additional resection. At this time, the evaluation unit 64 stores the warning to the user in the implementation information storage 84 as the implementation information.

The image analyzing device 3 detects a matter that has occurred during the implementation of the treatment as a treatment content. Specifically, the image analyzing device 3 detects a degree of bleeding and outputs implementation information including information indicating the degree of bleeding to the server device 2 after the end of the treatment. In addition, the image analyzing device 3 monitors the occurrence of perforation and outputs implementation information including information indicating whether or not perforation has occurred to the server device 2 after the treatment is completed. In the server device 2, the implementation information acquisition unit 44 stores the implementation information acquired from the start to the completion of the treatment in the implementation information storage 84 in association with the examination ID of the endoscopy and a treatment ID for identifying the treatment.

When the treatment tool is framed out from the endoscopic image, the image analyzing device 3 determines the end of the treatment and ends the counting of the treatment time. Note that the image analyzing device 3 may determine the end of the treatment and end the counting of the treatment time at the timing when the physician operates the release switch after the treatment tool 102 is framed out from the endoscopic image and the treated part is washed with water. Here, the treatment implementation end time is 13:19:52. Upon determination of the end of the treatment, the image analyzing device 3 provides the server device 2 with implementation information including the treatment time that has been count (time obtained by subtracting the implementation start time from the implementation end time) together with the treatment implementation end time.

In the server device 2, the implementation information acquisition unit 44 acquires the implementation information including the treatment time and the treatment end time, and stores the implementation information in the implementation information storage 84. The output processing unit 66 provides the treatment end time to the endoscope system 9 and ends the counting of the treatment time in the endoscope system 9.

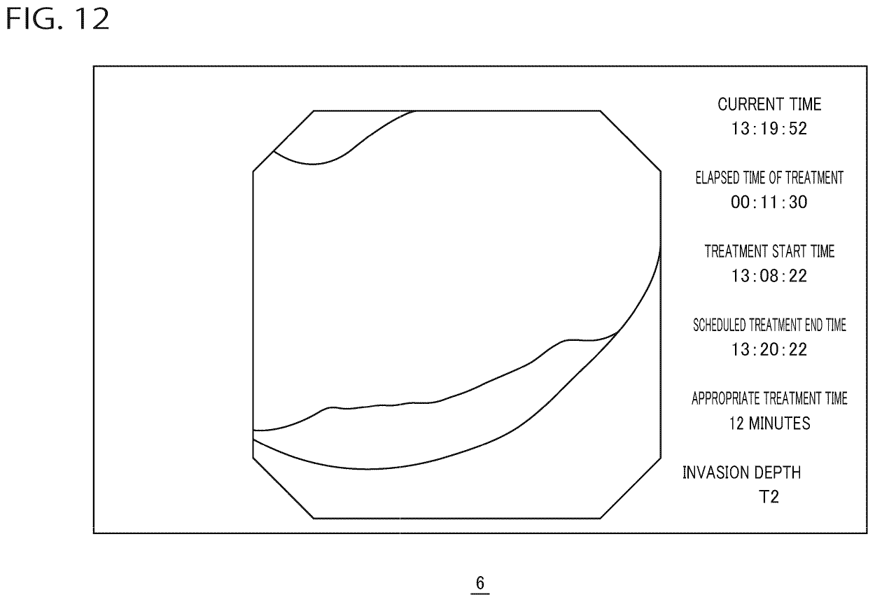

FIG. 12 shows an example of information displayed on the display device 6. Upon acquisition of the treatment end time from the output processing unit 66, the endoscopic observation device 5 ends the counting of the elapsed time of treatment. In this example, the time required for the treatment by the user is 11 minutes and 30 seconds, and is within 12 minutes which is the appropriate treatment time. This means that the user has completed the treatment within the appropriate time and has cleared the skill evaluation criterion related to the treatment time.

When the treatment ends, the evaluation unit 64 generates skill evaluation information evaluating the skill of the user who has performed the treatment based on the skill evaluation criterion set by the skill evaluation criterion setting unit 62 and the implementation information acquired by the implementation information acquisition unit 44. The evaluation unit 64 may generate the skill evaluation information based on the appropriate time included in the skill evaluation criterion and the treatment time included in the implementation information. In addition, the evaluation unit 64 may generate the skill evaluation information based on the skill evaluation criterion related to the resection line 100 and the resection edge information indicating the position of the resection edge at which the user has removed the lesion. The evaluation unit 64 according to the embodiment may score the treatment skills of the user by a deduction method from a maximum score (for example, 50 points). The master DB 88 records scores for deduction corresponding to the implementation information.

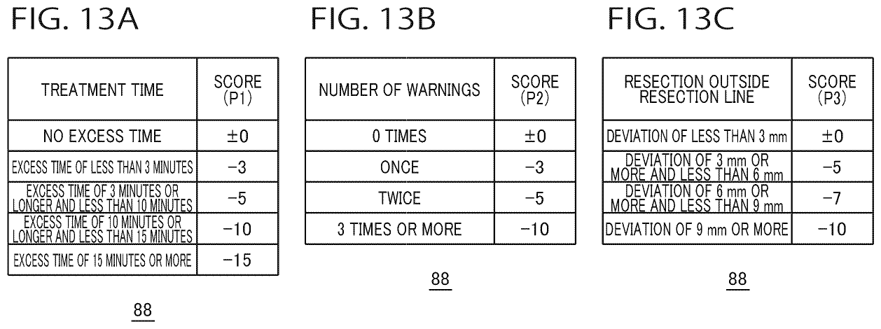

FIG. 13A shows an example of a table in which treatment time and deduction points are associated with each other. In this table, the excess time from the appropriate time, that is, (treatment time-appropriate time) and deduction points are associated, and to be specific, deduction points 0 are assigned to “no excess time”, deduction points 3 are assigned to “excess time of less than 3 minutes”, deduction points 5 are assigned to “excess time of 3 minutes or longer and less than 10 minutes”, deduction points 10 are assigned to “excess time of 10 minutes or longer and less than 15 minutes”, and deduction points 15 are assigned to “excess time of 15 minutes or more”.

FIG. 13B shows an example of a table in which the number of warnings and deduction points are associated with each other. As described above, the warning is output when the user removes within the resection line 100. In this table, deduction points 0 are assigned to “no warning”, deduction points 3 are assigned to “one warning”, deduction points 5 are assigned to “two warnings”, and deduction points 10 are assigned to “three times or more warnings”.

FIG. 13C shows an example of a table in which the amount of deviation when the outside of the resection line 100 is resected is associated with deduction points. This situation corresponds to a case where the user resects the living tissue more than necessary, and the deviation amount indicates the maximum distance from the resection line 100. In this table, deduction points 0 are assigned to “deviation of less than 3 mm”, deduction points 5 are assigned to “deviation of 3 mm or more and less than 6 mm”, deduction points 7 are assigned to “deviation of 6 mm or more and less than 9 mm”, and deduction points 10 are assigned to “deviation of 9 mm or more”.

The evaluation unit 64 derives the deduction points related to the skill evaluation criterion by performing the following steps (1) to (3).

(1) The evaluation unit 64 calculates (treatment time-appropriate time) with reference to the implementation information stored in the implementation information storage 84, and specifies a deduction point P1 corresponding to (treatment time-appropriate time) from the table shown in FIG. 13A.

(2) The evaluation unit 64 refers to the implementation information stored in the implementation information storage 84 to specify the number of times of warning output, and specifies a deduction point P2 corresponding to the number of times of warning output from the table shown in FIG. 13B.

(3) The evaluation unit 64 refers to the implementation information stored in the implementation information storage 84 and determines whether the resection edge from which the lesion is removed by the user is located outside the resection line 100. When the resection edge is located outside the resection line 100, the evaluation unit 64 specifies the maximum amount of deviation between the resection edge and the resection line 100 from the position coordinates of the resection edge and the position coordinates of the resection line 100, and specifies a deduction point P3 corresponding to the amount of deviation from the table shown in FIG. 13C.

After specifying the deduction points P1 to P3, the evaluation unit 64 calculates the skill score of the lesion treatment using the following calculation formula (4).

$$Skill score = 50 - ( P 1 + P 2 + P 3 ) \qquad ( 4 )$$

The evaluation unit 64 generates skill evaluation information from the calculated skill score.

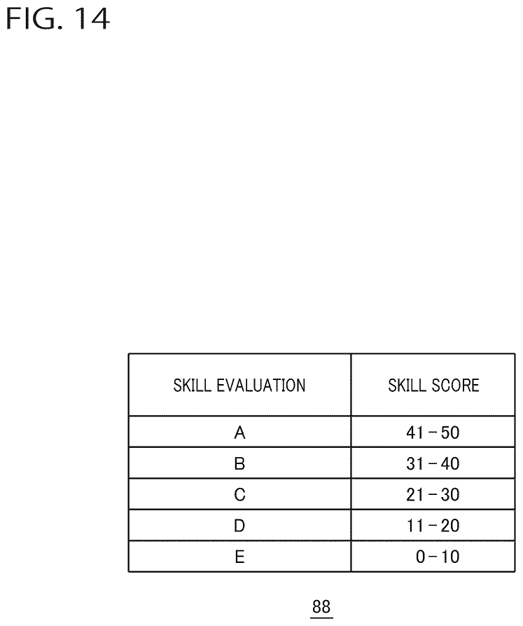

FIG. 14 shows an example of a table in which skill evaluation and skill scores are associated with each other. In the embodiment, the evaluation unit 64 evaluates the skills of the treatment in five ranks of ranks A to E from the skill score calculated for one treatment. In the embodiment, the ranks A to E are referred to as skill evaluation information, but the skill score itself may be referred to as skill evaluation information.

The evaluation unit 64 may generate the skill evaluation information based on one or both of the information related to bleeding and the information related to perforation.

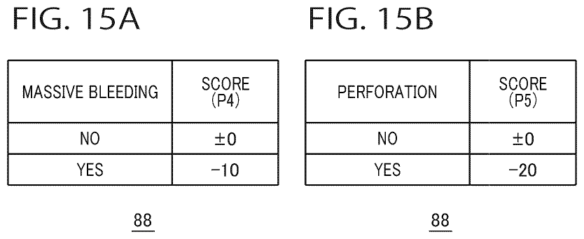

FIG. 15A shows an example of a table in which the presence or absence of massive bleeding is associated with deduction points. In this table, deduction points 0 are assigned to “no massive bleeding”, and deduction points 10 are assigned to “massive bleeding”.

FIG. 15B shows an example of a table in which the presence or absence of perforation is associated with deduction points. In this table, deduction points 0 are assigned to “no perforation”, and deduction points 20 are assigned to “perforation”.

The evaluation unit 64 performs the following steps (4) to (5) to derive the deduction points related to a bleeding situation and a perforation occurrence situation.

(4) The evaluation unit 64 refers to the implementation information stored in the implementation information storage 84 to determine whether or not there is massive bleeding, and specifies a deduction point P4 from the table shown in FIG. 15A.

(5) The evaluation unit 64 refers to the implementation information stored in the implementation information storage 84 to determine whether or not perforation has occurred, and specifies a deduction point P5 from the table shown in FIG. 15B.

After specifying the deduction points P1 to P5, the evaluation unit 64 calculates the skill score of the lesion treatment using the following calculation formula (5).

$$Skill score = 50 - ( P 1 + P 2 + P 3 + P 4 + P 5 ) \qquad ( 5 )$$

The evaluation unit 64 generates skill evaluation information from the calculated skill score with reference to the table shown in FIG. 14.

Here, when
deduction point P1: 0,
deduction point P2: 3,
deduction point P3: 7,
deduction point P4: 0, and
deduction point P5: 20,

the evaluation unit 64 calculates the skill score=20, and thus generates skill evaluation information of the rank D.

As described above, the evaluation unit 64 generates the skill evaluation information that evaluates the skills of the user based on the skill evaluation criterion and the implementation information stored in the implementation information storage 84. The evaluation unit 64 stores the generated skill evaluation information in the skill evaluation information storage 86 in association with the user ID, the examination ID, and the treatment ID. In the storage device 80, the skill evaluation information may be associated with the implementation information so that, for example, when an expert physician operates the terminal device 10b to confirm the skill evaluation information generated for a young physician, the implementation information can also be referred to.

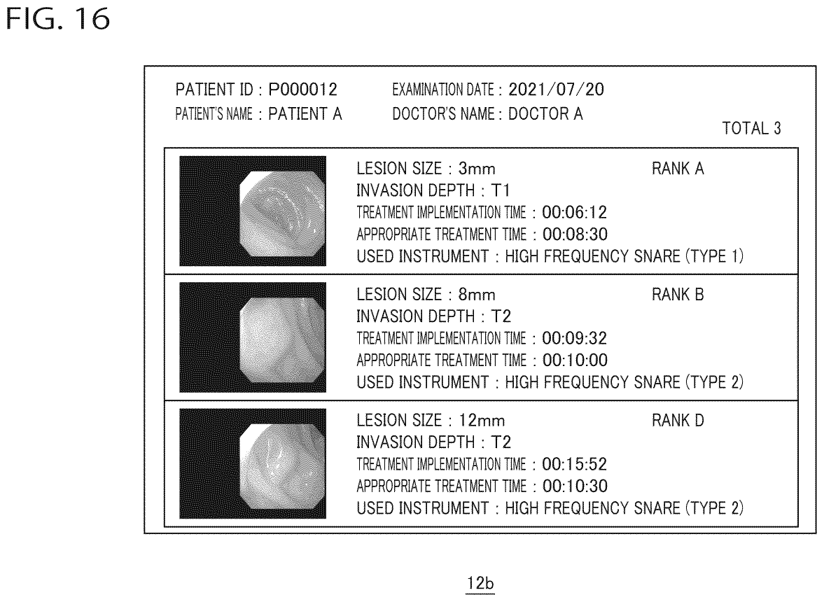

FIG. 16 shows an example of a screen displayed on the display device 12b. The screen shown in FIG. 16 includes information related to a treatment performed in one endoscopy. When an expert physician operates the information processing device 11b to input the examination ID of the endoscopy as a search condition, the output processing unit 66 in the server device 2 reads the skill evaluation information and the implementation information associated with the examination ID from the storage device 80 and provides the skill evaluation information and the implementation information to the information processing device 11b. In this example, a physician A performs three treatments in endoscopy, and the skill evaluation information for each of the three treatments is displayed in a form of the rank. By viewing the screen shown in FIG. 16, an expert physician can confirm the skill evaluation in a plurality of treatments performed in endoscopy.

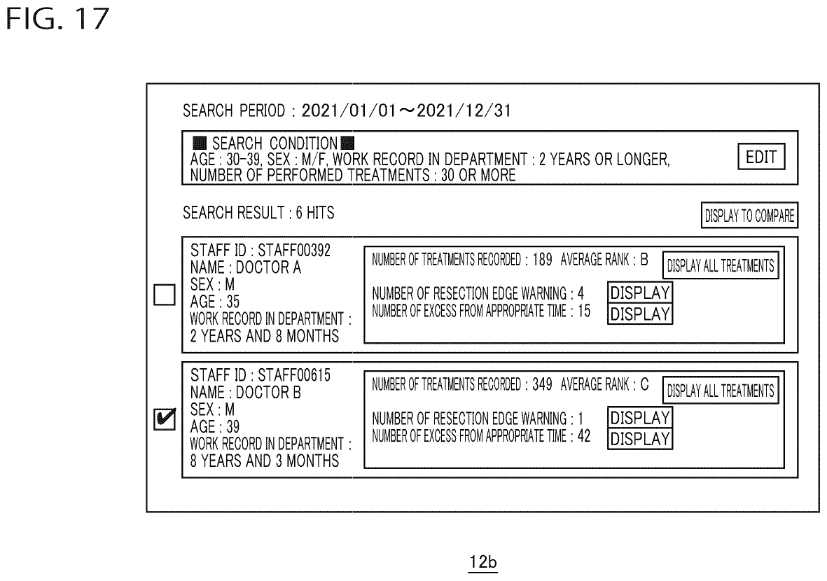

FIG. 17 shows another example of the screen displayed on the display device 12b. The screen shown in FIG. 17 includes information related to treatment records of the user corresponding to the search condition. When an expert physician operates the information processing device 11b to input information related to the user as a search condition, the output processing unit 66 receives the search condition and reads out all the implementation information and skill evaluation information of the corresponding user from the storage device 80 based on the search condition. The output processing unit 66 performs statistical processing on the implementation information and the skill evaluation information that have been read, and provides a result of the statistical processing to the information processing device 11b. In this example, average values of skill evaluation information of users hitting the search condition is displayed as “average rank”. The expert physician can simultaneously confirm the skill evaluation information associated with each of the plurality of users by viewing the screen shown in FIG. 17.

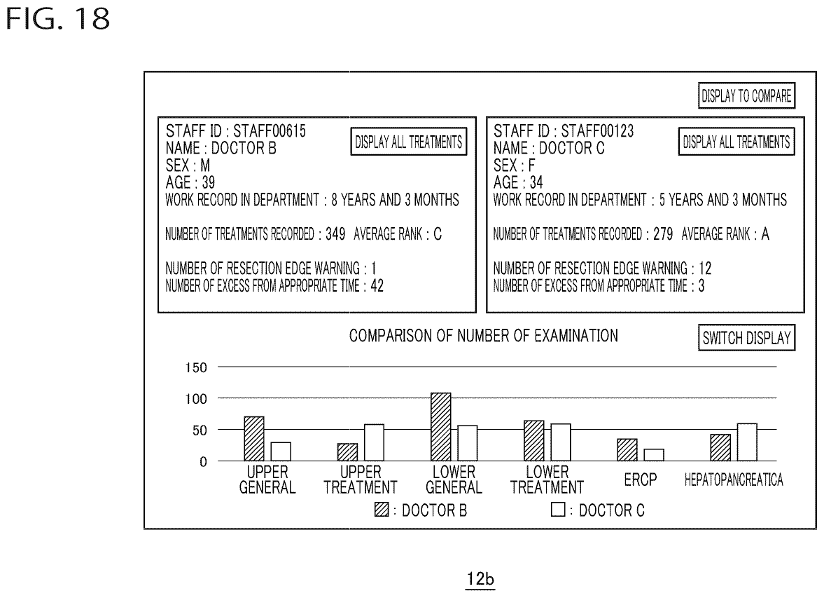

FIG. 18 shows another example of the screen displayed on the display device 12b. The screen shown in FIG. 18 includes information related to treatment records of two physicians B and C. When an expert physician operates the information processing device 11b to designate two users, the output processing unit 66 reads implementation information and skill evaluation information of the two users from the storage device 80, performs statistical processing, and provides a result of the statistical processing to the information processing device 11b. By viewing the screen shown in FIG. 18, an expert physician can simultaneously confirm and compare the skill evaluation information associated with the physician B and the physician C.

The present disclosure has been described above based on the plurality of embodiments. It is to be understood by those skilled in the art that these embodiments are examples, that various modifications can be made to combinations of the components and the processes, and that such modifications are also within the scope of the present disclosure. In the embodiments, the terminal device 10a is provided with various types of information from the server device 2, but may be provided from the image analyzing device 3.

What is claimed is:

1. A medical support system comprising:
one or more processors having hardware, wherein
the one or more processors are configured to:
acquire lesion information including at least one of a size and an invasion depth of a lesion detected from an endoscopic image by a computer; and
set a skill evaluation criterion related to a treatment to be performed on the detected lesion based on the lesion information.

2. The medical support system according to claim 1, wherein
the one or more processors are configured to:
set the skill evaluation criterion including an appropriate time required to perform the treatment on the lesion.

3. The medical support system according to claim 1, wherein
the one or more processors are configured to:
set the skill evaluation criterion including a resection line for resecting the lesion.

4. The medical support system according to claim 1, wherein
the one or more processors are configured to:
acquire implementation information indicating a treatment content performed on the lesion by a user; and
generate skill evaluation information evaluating skills of the user who has performed the treatment based on the skill evaluation criterion and the implementation information.

5. The medical support system according to claim 1, wherein
the one or more processors are configured to:
acquire the lesion information including a differential diagnosis result of the lesion; and
set the skill evaluation criterion based on the differential diagnosis result of the lesion.

6. The medical support system according to claim 1, wherein
the one or more processors are configured to:
acquire treatment tool information related to a type of a treatment tool to be used; and
set the skill evaluation criterion based on the lesion information and the treatment tool information.

7. The medical support system according to claim 6, wherein
the one or more processors are configured to:
acquire information indicating a past treatment content where the same type of the treatment tool was used in the past treatment; and
set the skill evaluation criterion based on the information indicating the past treatment content.

8. The medical support system according to claim 1, wherein
the one or more processors are configured to:
acquire patient information including at least one of information related to a residue, information related to an inflammation, and information related to age; and
set the skill evaluation criterion based on the lesion information and the patient information.

9. The medical support system according to claim 4, wherein
the one or more processors are configured to:
set the skill evaluation criterion including an appropriate time required to perform the treatment on the lesion;
acquire implementation information including a treatment time taken by a user to treat the lesion; and
generate the skill evaluation information based on the appropriate time included in the skill evaluation criterion and the treatment time included in the implementation information.

10. The medical support system according to claim 9, wherein
the one or more processors are configured to:
acquire the lesion information including the size of the lesion, the invasion depth of the lesion, and a differential diagnosis result of the lesion;
acquire treatment tool information related to a type of a treatment tool to be used;
set the skill evaluation criterion including the appropriate time based on the lesion information and the treatment tool information; and
generate the skill evaluation information based on the appropriate time included in the skill evaluation criterion and the treatment time included in the implementation information.

11. The medical support system according to claim 9, wherein
the one or more processors are configured to:
display, while the treatment of the lesion is performed, an image captured in endoscopy, information indicating the appropriate time, and time after starting the treatment of the lesion, on one or more display devices.

12. The medical support system according to claim 6, wherein
the one or more processors are configured to:
display, on a display device, before the treatment of the lesion is started, information indicating an appropriate time that has been set based on a type of a treatment tool estimated to be used; and
display, on the display device, when the type of the treatment tool to be used is different from the type of the treatment tool that has been estimated, information indicating the appropriate time set based on the type of the treatment tool to be used.

13. The medical support system according to claim 3, wherein
the one or more processors are configured to:
acquire the lesion information including contour information indicating a position of a contour of the lesion;
set the skill evaluation criterion related to the resection line of the lesion based on the contour information of the lesion;
acquire implementation information including resection edge information indicating a position of a resection edge at which a user has resected the lesion; and
generate skill evaluation information evaluating skills of the user who has resected the lesion based on the skill evaluation criterion and the resection edge information.

14. The medical support system according to claim 13, wherein
the one or more processors are configured to:
output a warning when the resection edge at which the user has resected the lesion is within the resection line; and
generate the skill evaluation information based on a number of times the warning is output.

15. The medical support system according to claim 13, wherein
the one or more processors are configured to:
generate the skill evaluation information based on a distance between the resection edge and the resection line when the resection edge at which the user has resected the lesion is outside the resection line.

16. The medical support system according to claim 4, wherein
the one or more processors are configured to:
acquire the implementation information including information related to bleeding at a time of resection of the lesion; and
generate the skill evaluation information based on the information related to the bleeding.

17. The medical support system according to claim 4, wherein
the one or more processors are configured to:
acquire the implementation information including information related to perforation at a time of resection of the lesion; and
generate the skill evaluation information based on the information related to the perforation.

18. The medical support system according to claim 4, further comprising:
a storage device structured to store the skill evaluation information in association with the user.

19. A medical support method comprising:
acquiring lesion information including at least one of a size and an invasion depth of a lesion detected from an endoscopic image by a computer; and
setting a skill evaluation criterion related to a treatment to be performed on the detected lesion based on the lesion information.

20. A recording medium having a program recorded therein, the program causing a computer to achieve:
a function of acquiring lesion information including at least one of a size and an invasion depth of a lesion detected from an endoscopic image by another computer; and
a function of setting a skill evaluation criterion related to a treatment to be performed on the detected lesion based on the lesion information.
